(19) United States
(12) Patent Application Publication
(10) Pub. No.: US 2025/0177655 A1
(43) Pub. Date: Jun. 5, 2025

(54) MEDICAMENT DELIVERY DEVICE

(71) Applicant: SHL Medical AG, Zug (CH)

(72) Inventor: Nurettin Ali, Deerfield Beach, FL (US)

(21) Appl. No.: 18/836,906

(22) PCT Filed: Jan. 25, 2023
(86) PCT No.: PCT/EP2023/051795

§ 371 Date: Aug. 8, 2024

Related U.S. Application Data
(60) Provisional application No. 63/307,698, filed on Feb. 8, 2022.

(30) Foreign Application Priority Data
Apr. 20, 2022 (EP) 22169059.7

(51) Int. Cl. A61M 5/315

(52) U.S. Cl. CPC A61M 5/31515; A61M 5/3158

(57) ABSTRACT

The present disclosure provides medicament delivery device, comprising a housing, a plunger rod, a tubular extension member, a U-bracket, a first energy accumulation member, and a medicament container, said U-bracket comprising a first longitudinally extending leg and a second longitudinally extending leg, a distal end of the first longitudinally extending leg being connected to a distal end of the second longitudinally extending leg by a transversal end portion of the U-bracket, a proximal end of the first longitudinally extending leg being bent radially outward, thereby defining a first bent portion, the U-bracket comprising a third longitudinally extending leg, a distal end of the third longitudinally extending leg being attached to the first bent portion, a proximal end of the third longitudinally extending leg comprising a radially extending portion, the radially extending portion of the third longitudinally extending leg defining a first radial foot, the first radial foot supporting the medicament container by abutting a flange of the medicament container.

### CROSS-REFERENCE TO RELATED APPLICATIONS

The present application is a U.S. National Phase Application pursuant to 35 U.S.C. § 371 of International Application No. PCT/EP2023/051795 filed Jan. 25, 2023, which claims priority to (i) U.S. Provisional Patent Application No. 63/307,698 filed Feb. 8, 2022, and (ii) European Patent Application No. 22169059.7 filed Apr. 20, 2022. The entire disclosure contents of these applications are herewith incorporated by reference into the present application.

### TECHNICAL FIELD

The present application relates to U-brackets for medicament delivery devices, and particularly to U-brackets for supporting a medicament container of a medicament delivery device.

### BACKGROUND

Regarding medicament delivery devices having a medicament container assembled therein, it is often a desire that the medicament container is supported such that it may not rattle or move inside the housing of the medicament delivery device. Due to tolerance variations of both the medicament container and the medicament delivery device as such, any support for a medicament container should preferably be flexible and possibly also resilient in order to handle forces exerted on the medicament delivery device and thus the medicament container if it e.g. is dropped onto a hard surface.

Since those medicament delivery devices are designed for patients without formal medical training and operations of those medicament delivery devices might take place outside of a place of professional health/medical care, e.g. hospital, clinic or heath centres, it is also useful if the medicament delivery devices provide feedback at different stages of a medicament delivery operation.

Whilst both of these functions are known in existing medicament delivery devices, it has been appreciated that further developments could be made to improve tolerance management and user feedback.

### BRIEF DESCRIPTION

A first aspect concerns a medicament delivery device, comprising:
a housing having a proximal end and a distal end, a plunger rod received by the housing and axially displaceable from an initial position to a final position relative to the housing, a tubular extension member received by the housing and arranged to receive the plunger rod, a U-bracket received by the tubular extension member, the plunger rod being arranged to be received by the U-bracket, a first energy accumulation member arranged to bias the plunger rod in a proximal direction towards the proximal end and to bias the U-bracket in a distal direction which is opposite to the proximal direction, a medicament container provided with a needle, a plunger being arranged to move into the medicament container by linear displacement of the plunger rod to thereby expel medicament through the needle,
wherein said U-bracket comprises a first longitudinally extending leg and a second longitudinally extending leg, a distal end of the first longitudinally extending leg being connected to a distal end of the second longitudinally extending leg by a transversal end portion of the U-bracket, a proximal end of the first longitudinally extending leg being bent radially outward, thereby defining a first bent portion, the U-bracket comprising a third longitudinally extending leg, a distal end of the third longitudinally extending leg being attached to the first bent portion, a proximal end of the third longitudinally extending leg comprising a radially extending portion, the radially extending portion of the third longitudinally extending leg defining a first radial foot, the first radial foot supporting the medicament container by abutting a flange of the medicament container.

In addition to being able to provide a feedback function, for example for indicating a delivery operation of a medicament delivery device, the U-bracket has a structure that enables supporting the medicament container in a steady state inside the medicament delivery device. The U-bracket can hold the medicament container in a steady state inside the medicament delivery device. The bent portion of the radially outward bent leg can provide a bouncy feature to the U-bracket and can increase the diameter of the U-bracket.

Optionally, the second longitudinally extending leg at the proximal end is bent radially outward defining a second bent portion. Optionally, the U-bracket comprises a fourth longitudinally extending leg, wherein a distal end of the fourth longitudinally extending leg is attached to the second bent portion, wherein a proximal end of the fourth longitudinally extending leg comprises a radially extending portion. The radially extending portion defines a second radial foot, the second radial foot being configured to support the medicament container by abutting a flange of the medicament container.

The first and second radially outward bent portions can provide a symmetrically aligned support for the medicament container.

The first and second radial leg can provide a bouncy contact between the U-bracket and the flanges of the medicament container, thereby holding the medicament container safely in place.

Optionally, the U-bracket is made of metal. Optionally, the U-bracket is made of sheet metal. Optionally, the U-bracket is made of wire. The wire may be metal. Optionally, the U-bracket is made of plastic. The different materials can provide options regarding elasticity, sturdiness and manufacturing considerations, for example to help with cost or with ease of manufacture.

Optionally, the first longitudinally extending leg is parallel to the second longitudinally extending leg.

Optionally, the distance between the distal end of the first longitudinally extending leg and the distal end of the second longitudinally extending leg is less than the distance between the proximal end of the first longitudinally extending leg and the proximal end of the second longitudinally extending leg.

Optionally, the distal end of the third longitudinally extending leg is further from the axis than the proximal end of the third longitudinally extending leg.

Optionally, the U-bracket is flexible.

Optionally, the medicament delivery device is a single use device.

Optionally, the medicament delivery device is a multiple use device.

The present disclosure is applicable to a number of medical delivery devices, including, but not limited to, devices that automatically, semi-automatically or manually deliver a dose of medicament through injection.

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component. The circumferential direction describes a direction extending around the axis, so around the circumference of a device or component, and the radial direction extends perpendicular to the axis. Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

The term automatic in conjunction with a medical delivery device means that the medicament delivery device is able to perform the injection without the user of the device delivering the force needed to expel the medicament during delivery. The force is delivered automatically by a resilient member like a spring or an electric motor or any other suitable means for providing the delivery force.

These and other aspects and advantages of the present invention will become apparent from the following detailed description and from the accompanying drawings.

### BRIEF DESCRIPTION OF THE DRAWINGS

Embodiments of the invention will now be described by way of example only and with reference to the accompanying drawings, in which:

FIG. 1 shows a cross sectional view of a rear subassembly of a medicament device.

FIG. 2 shows a perspective view of a U-bracket, a plunger rod, a drive spring and part of a syringe.

FIG. 3 shows a medicament delivery device.

FIG. 4 shows an exploded view of the medicament delivery device of FIG. 3.

FIG. 5 shows the tubular extension member with flexible tongues engaging with a radial opening of the plunger rod.

### DETAILED DESCRIPTION

The present application is directed to a U-bracket for supporting a medicament container of a medicament delivery device, the U-bracket extending longitudinally along a longitudinal axis from a proximal end to a distal end. The U-bracket comprises a first longitudinally extending leg and a second longitudinally extending leg, wherein a distal end of the first longitudinally extending leg is connected to a distal end of the second longitudinally extending leg by a transversal end portion of the U-bracket, and wherein a proximal end of the first longitudinally extending leg is bent radially outward, thereby defining a first bent portion. The U-bracket comprises a third longitudinally extending leg, wherein a distal end of the third longitudinally extending leg is attached to the first bent portion, and wherein a proximal end of the third longitudinally extending leg comprises a radially extending portion.

The U-bracket will now be described more fully hereinafter with reference to the accompanying drawings.

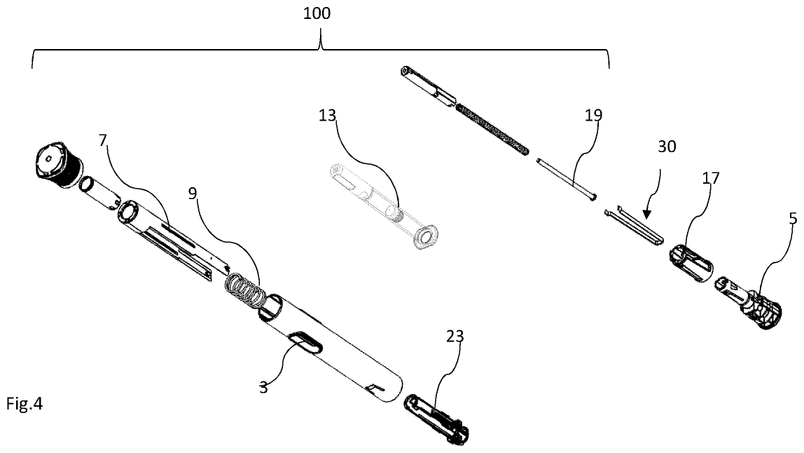

The medicament delivery device 100 as shown in FIG. 3 has a housing 3 having a proximal end P and a distal end D, a plunger rod 12 received by the housing 3 and axially displaceable from an initial position to a final position relative to the housing, a tubular extension member (in this case a rear cap) 5 received by the housing 3 and arranged to receive the plunger rod 12, a U-bracket 30 received by the tubular extension member 5, wherein the plunger rod 12 is arranged to be received by the U-bracket, and a first energy accumulation member 121 arranged to bias the plunger rod 12 in a proximal direction towards the proximal end P and to bias the U-bracket in a distal direction which is opposite to the proximal direction.

The plunger rod 12, which in the initial position is in a pre-tensioned state, is arranged to flex or press the U-bracket 30 radially outward towards the tubular extension member thereby engaging the U-bracket with the tubular extension member 5. To this end, the plunger rod 12 has a first section with a first thickness or dimension, which section presses the U-bracket 30, which in the initial position is mounted around the plunger rod 12, radially outwards. The U-bracket is thereby engaged with the tubular extension member 5, retaining the distally biased U-bracket in a fixed axial position relative to the housing 3. In this axial position, the U-bracket is distanced from a distal inner surface 51 of the tubular extension member 5.

Prior to use, the plunger rod 12 is arranged axially fixed in the initial position. When the medicament delivery device 100 is activated, i.e. when a user initiates medicament delivery, the plunger rod 12 is released from the initial position and is pressed in the proximal direction by the energy accumulation member 121. When the plunger rod 12 has been displaced a distance corresponding to a distance necessary for completion of medicament expulsion, the contact between a first section of the plunger rod 12 and the U-bracket will cease. The plunger rod 12 and the U-bracket are thus designed such that the axial overlap of the first section and U-bracket in the initial position of the plunger rod 12 corresponds to, i.e. is equal to or essentially equal to, said distance. Hence, the first section only supports the U-bracket 30 during axial displacement of the plunger rod 12 distance corresponding to a distance necessary for completion of medicament expulsion. When the plunger rod 12 has been displaced by an amount corresponding to this distance, the U-bracket 30 will be allowed to flex radially inwards towards its radially unbiased state. The U-bracket will thereby disengage from the tubular extension member 5. The U-bracket 30, which is biased in the distal direction by the first energy accumulation member 121 will thereby no longer be retained by the tubular extension member 5 and will therefore be thrown towards the distal inner surface 51 of the tubular extension member. This results in an audible “click” sound, and also provides the user with a tactile sensation. Alternatively, the U-bracket could be arranged so that the feedback is provided at a different point, for example at the start of medicament delivery.

The medicament delivery device 100 may furthermore comprise a medicament delivery member cover received by and rotationally interlocked with the housing 3, and a tubular rotator arranged to receive the plunger rod 12 and the tubular extension member 5. The medicament delivery member cover is displaceable axially between an extended position and a retracted position relative to the housing. The medicament delivery member cover is biased towards the extended position.

The tubular rotator is arranged to interact with the medicament delivery member and has a guide structure arranged to convert linear motion of the medicament delivery member to rotational motion of the tubular rotator.

FIG. 3 shows an example of a medicament delivery device 100. The medicament delivery device 100 comprises a housing 3 having a proximal end P and a distal end D, a tubular extension member 5, for example a rear cap, mounted at the distal end of the housing 3, and a medicament delivery member cover, e.g. a needle cover, arranged to be received by the housing 3 and arranged to be biased in the proximal direction.

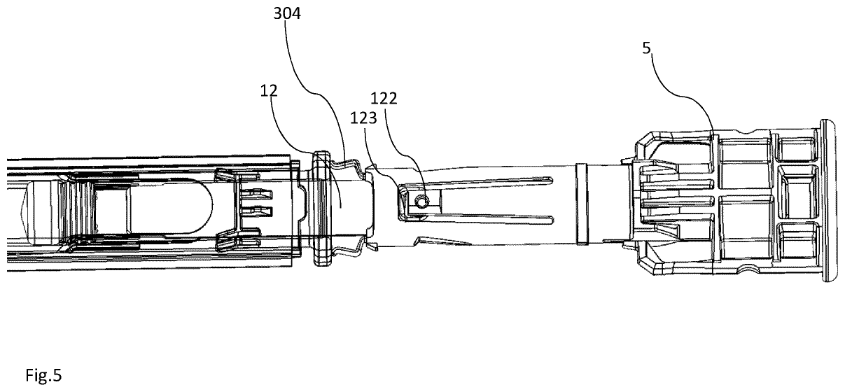

FIG. 4 depicts an exploded view of the medicament delivery device 100. The medicament delivery device 100 further comprises a plunger rod 12 which is arranged to be biased towards the proximal end P, a plunger 13, a first resilient member, i.e. an energy accumulation member 121, arranged to bias the plunger rod 12 in the proximal direction, which first energy accumulation member 121 may be a spring for example, a second resilient member or energy accumulation member 9, arranged to bias the medicament delivery member cover 7 in the proximal direction, a tubular rotator 17 arranged to receive the plunger rod 12 and the first energy accumulation member 121, a tubular extension member 5 which is axially and rotationally fixed relative to the housing 3, a tubular operation member 17, in the following referred to as tubular rotator 17, arranged to receive a portion of the rear end cap 7 and the plunger rod 12, a rod 19 which the first energy accumulation member 121 is arranged to receive, and a U-bracket 30. The exemplified medicament delivery device 100 furthermore comprises a medicament container holder 23 and a medicament container 20 provided with a needle. The plunger 13 is arranged to move into the medicament container 20 by linear displacement of the plunger rod 12, to thereby expel medicament through the needle.

The plunger rod 12, the tubular extension member 5, the tubular operation member 17, the energy accumulation member 121 and the U-bracket 30 form an automatic feedback mechanism, or simply a feedback mechanism, for the medicament delivery device 100. The feedback mechanism is arranged to notify a user of a start of expulsion of medicament from the medicament container 20.

As shown in FIG. 5, the plunger rod 12 has a radial opening 123, and the tubular extension member 5 which is arranged to receive the plunger rod 12 has a corresponding flexible tongue 122 flexible in the radial direction and arranged to engage with the opening 123. The tubular rotator 17 is arranged to receive a portion of the tubular extension member 5, in particular that portion which comprises the flexible tongue 122.

The tubular rotator 17 is rotatable from an initial rotational position to a final rotational position. In its initial rotational position, the tubular rotator 17 is arranged to hold the flexible tongue 122 in engagement with the opening 123, preventing the plunger rod 12 from axial displacement. When the tubular rotator 17 is rotated, the inner structure of the tubular rotator 17 is designed such that it will allow the flexible tongue 122 to flex radially outwards to disengage from the plunger rod 12. The plunger rod 12, which is biased in the proximal direction, is thereby displaced axially and medicament administration is thus commenced as the plunger rod 12 pushes the plunger 13 inside the medicament container 20.

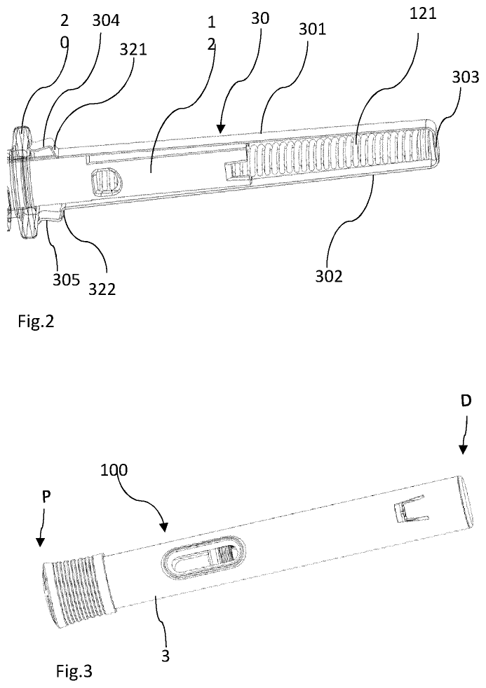

FIG. 2 shows a first example of a plunger rod 12 and a U-bracket 30. The U-bracket 30 has a distal transversal end portion 303 and two longitudinally extending legs 301,302 connected by the distal transversal end portion 303. Preferably the first and the second longitudinally extending legs 301, 302 are parallel to each other.

The plunger rod 12 has a hollow body and a distal end opening. The plunger rod 12 is arranged to receive the first energy accumulation member 121 in the distal end opening, such that the first energy accumulation member 121 extends in the distal direction out from the distal end opening. The plunger rod 12 is arranged to retain the proximal end of the first energy accumulation member 121 in the hollow body to enable biasing by means of the first energy accumulation member 121. The U-bracket 30 is arranged to receive the plunger rod 12, from the distal end of the plunger rod 12, and the first energy accumulation member 121 is arranged to be compressed between the distal transverse end portion 303 and a stop inside the plunger rod 12 when the plunger rod 12 is in the initial position. In the initial position of the plunger rod 12, the radial feet 32, 34 are in engagement with the flanges of the medicament container 20.

The U-bracket 30 may, in its end position once it has collided with the distal inner surface 51, engage with the tubular extension member 5. The plunger rod 12 moves past the U-bracket 30 during medicament expulsion, in particular the medicament delivery device 100 typically reaches a final state, after medicament delivery has been finalised, where the plunger rod 12 has moved past the U-bracket 30 in the proximal direction.

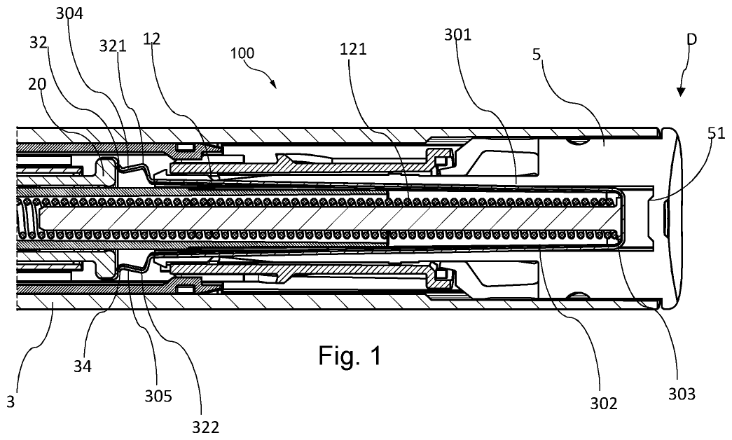

FIG. 1 illustrates a rear portion of a medicament delivery device 100 that comprises a housing 3 showing the distal end D, and extending along a longitudinal axis L. The plunger rod 12, which is received in the housing 3, is also shown, as is the first energy accumulation member 121, which is preferably a resilient member as a spring received by the plunger rod 12. As shown in FIG. 2, the U-bracket 30 extends around the plunger rod 12 and first energy accumulation member 121.

One end of the first energy accumulation member 121 is supported by an inner support surface of the transversal end portion 303 of the U-bracket 30 at its distal end and the other end of the drive member is supported by an inner shoulder of the plunger rod 12 at its proximal end (not shown in the figures). The first energy accumulation member 121 is configured to proximally bias the plunger rod 12 in relation to the housing 3 and the U-bracket 30.

As shown in FIG. 2 the U-bracket 30 extends longitudinally along a longitudinal axis from the proximal end P to the distal end D, the U-bracket 30 comprises a first longitudinally extending leg 301 and a second longitudinally extending leg 302, wherein the distal end of the first longitudinally extending leg 301 is connected to the distal end of the second longitudinally extending leg 302 by the transversal end portion 303. The proximal end of the first longitudinally extending leg 301 is bent radially outward, thereby defining a first bent portion 321. Attached to this first bent portion is a third longitudinally extending leg 304, The third longitudinally extending leg 304 extends longitudinally and comprises a distal end and a proximal end, wherein the proximal end of the third longitudinally extending leg 304 comprises a radially extending portion and the distal end of the third longitudinally extending leg 304 is attached to the first longitudinally extending leg 301.

In one example the U-bracket comprises the first longitudinally extending leg 301 and the second longitudinally extending leg 302, wherein the distal end of the first longitudinally extending leg 301 is connected to the distal end of the second longitudinally extending leg 302 by the transversal end portion 303. The proximal end of the first longitudinally extending leg 301 is bent radially outward, thereby defining a first bent portion 321. Attached to this first bent portion is a third longitudinally extending leg 304. The third longitudinally extending leg 304 extends longitudinally and comprises a distal end and a proximal end, wherein the proximal end of the third longitudinally extending leg 304 comprises a radially extending portion and the distal end of the third longitudinally extending leg 304 is attached to the first longitudinally extending leg 301. The third longitudinally extending leg 304, which extends from the first outwardly bent portion 321, has a slightly inwardly bent form. In other words, the distal end of the third longitudinally extending leg 304 is further from the axis than the proximal end of the third longitudinally extending leg 304. The radially extending portion of the third longitudinally extending leg 304 defines a first radial foot 32, the first radial foot 32 supports the medicament container 20 by abutting the flange of the medicament container 20. The U-bracket 30 further comprises at the proximal end of the second longitudinally extending leg 302 a radially extending portion defining a second radial foot 34, the second radial foot 34 supports the medicament container 20 by abutting the flange of the medicament container 20.

In another example the U-bracket comprises the first longitudinally extending leg 301 and the second longitudinally extending leg 302, wherein the distal end of the first longitudinally extending leg 301 is connected to the distal end of the second longitudinally extending leg 302 by the transversal end portion 303. The proximal end of the first longitudinally extending leg 301 is bent radially outward, thereby defining a first bent portion 321. Attached to this first bent portion is a third longitudinally extending leg 304. The third longitudinally extending leg 304 extends longitudinally and comprises a distal end and a proximal end, wherein the proximal end of the third longitudinally extending leg 304 comprises a radially extending portion and the distal end of the third longitudinally extending leg 304 is attached to the first longitudinally extending leg 301. The third longitudinally extending leg 304, which extends from the first outwardly bent portion 321, has a slightly inwardly bent form. In other words, the distal end of the third longitudinally extending leg 304 is further from the axis than the proximal end of the third longitudinally extending leg 304. The radially extending portion of the third longitudinally extending leg 304 defines a first radial foot 32, the first radial foot 32 supports the medicament container 20 by abutting the flange of the medicament container 20. The second longitudinally extending leg 302 at the proximal end is bent radially outward defining a second bent portion 322, and the U-bracket 30 comprises at the proximal end a fourth longitudinally extending leg 305 extending proximally from the second bent portion 322. The fourth longitudinally extending leg 305, which extends from the second outwardly bent portion 322, has a slightly inwardly bent form. In other words, the distal end of the fourth longitudinally extending leg 305 is further from the axis than the proximal end of the fourth longitudinally extending leg 305. The fourth longitudinally extending leg 305 comprises at the proximal end a radially extending portion defining a second radial foot 34, the second radial foot 34 supports the medicament container 20 by abutting a flange of the medicament container 20.

In one example the U-bracket 30 is made of metal, for example sheet metal. In one example the U-bracket 30 is made of wire. In one example the U-bracket 30 is made of plastic, which may have the same thermal expansion as the other plastic parts.

In one example the U-bracket has a slightly conical shape, the U-bracket widens towards the proximal end, or in other words the distance between the distal end of the first longitudinally extending leg 301 and the distal end of the second longitudinally extending leg 302 is less than the distance between the proximal end of the first longitudinally extending leg 301 and the proximal end of the second longitudinally extending leg 302.

FIG. 1 shows a sub-assembly comprising a U-bracket 30 as described above and a plunger rod 12 arranged to be biased towards the proximal end of the medicament delivery device. The sub-assembly further comprises an energy accumulating member 121 arranged to be received inside the plunger rod 12 for biasing the plunger rod in the proximal direction.

In one example the sub-assembly is pre-assembled as a modular rear sub-assembly such that it can be easily attached to a second sub-assembly, namely a front sub-assembly. For example, different front sub-assemblies can be combined with the same rear sub-assembly, which has manufacturing advantages, for example in terms of cost or ease of manufacturing.

Another sub-assembly comprises an energy accumulating member which stepwise biases the plunger rod or comprises further a resetting mechanism for the energy accumulating member in order to reset it from the final to the initial position of the energy accumulating member. In this way, the sub assembly can provide the rear sub-assembly for a multi-dose or multi-use medicament delivery device.

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the figures.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

Further, in the following description, the wording medicament delivery device will be used. In this context, the term medicament delivery device may include various devices capable of delivering certain doses of medicament to a user. The medicament delivery devices may be either disposable or re-usable.

Although the medical device described below is a medicament delivery device configured as a disposable single-use, pen-type injector, such as an auto-injector, any other type of single-use automatic medicament delivery device could incorporate the feedback mechanisms of the present disclosure, including, but not limited to, inhalers or eye dispensers. Likewise, the medical device may be a training device that replicates a medicament delivery device.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a' (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-12 (canceled)

13. A medicament delivery device, comprising:
a housing having a proximal end and a distal end;
a plunger rod received by the housing and axially displaceable from an initial position to a final position relative to the housing,
a tubular extension member received by the housing and arranged to receive the plunger rod
a U-bracket received by the tubular extension member, the plunger rod being arranged to be received by the U-bracket,
a first energy accumulation member arranged to bias the plunger rod in a proximal direction towards the proximal end and to bias the U-bracket in a distal direction which is opposite to the proximal direction; and
a medicament container provided with a needle, a plunger being arranged to move into the medicament container by linear displacement of the plunger rod, to thereby expel medicament through the needle,
wherein said U-bracket comprises a first longitudinally extending leg and a second longitudinally extending leg,
a distal end of the first longitudinally extending leg being connected to a distal end of the second longitudinally extending leg by a transversal end portion of the U-bracket,
a proximal end of the first longitudinally extending leg being bent radially outward, thereby defining a first bent portion,
the U-bracket comprising a third longitudinally extending leg, a distal end of the third longitudinally extending leg being attached to the first bent portion, a proximal end of the third longitudinally extending leg comprising a radially extending portion,
the radially extending portion of the third longitudinally extending leg defining a first radial foot, the first radial foot supporting the medicament container by abutting a flange of the medicament container.

14. The medicament delivery device of claim 13, wherein a proximal end of the second longitudinally extending leg is bent radially outward, thereby defining a second bent portion,
the U-bracket comprising a fourth longitudinally extending leg,
a distal end of the fourth longitudinally extending leg being attached to the second bent portion, wherein a proximal end of the fourth longitudinally extending leg comprises a radially extending portion,
the radially extending portion defining a second radial foot, the second radial foot supporting the medicament container by abutting the flange of the medicament container.

15. The medicament delivery device of claim 13, wherein the U-bracket is made of metal.

16. The medicament delivery device of claim 13, wherein the U-bracket is made of sheet metal.

17. The medicament delivery device of claim 13, wherein the U-bracket is made of wire.

18. The medicament delivery device of claim 13, wherein the U-bracket is made of plastic.

19. The medicament delivery device of claim 13, wherein the first longitudinally extending leg is parallel to the second longitudinally extending leg.

20. The medicament delivery device of claim 13, wherein the distance between the distal end of the first longitudinally extending leg and the distal end of the second longitudinally extending leg is less than the distance between the proximal end of the first longitudinally extending leg and the proximal end of the second longitudinally extending leg.

21. The medicament delivery device of claim 13, wherein the distal end of the third longitudinally extending leg is further from the axis than the proximal end of the third longitudinally extending leg.

22. The medicament delivery device of claim 13, wherein the U-bracket is flexible.

23. The medicament delivery device of claim 13, wherein the medicament delivery device is a single use device.

24. The medicament delivery device of claim 13, wherein the medicament delivery device is a multiple use device.
